(19) United States
(12) Patent Application Publication
(10) Pub. No.: US 2026/0008773 A1
(43) Pub. Date: Jan. 8, 2026

(54) DIHYDROMYRICETIN (DHM) DERIVATIVE AND PREPARATION METHOD AND USE THEREOF

(71) Applicant: HONGKONG CAREFREEING BIO-TECH CO., LIMITED, HONG KONG (CN)

(72) Inventor: Qingrui MENG, HONG KONG (CN)

(73) Assignee: HONGKONG CAREFREEING BIO-TECH CO., LIMITED, HONG KONG (CN)

(21) Appl. No.: 19/318,388

(22) Filed: Sep. 4, 2025

(30) Foreign Application Priority Data
Jul. 4, 2024 (CN) 202410891587.3

(51) Int. Cl. C07D 405/06; A61K 31/352; A61K 31/4025; A61K 31/453; A61K 31/496; A61P 25/28; A61P 37/02; A61P 43/00; C07D 311/30

(52) U.S. Cl. CPC C07D 405/06; A61K 31/352; A61K 31/4025; A61K 31/453; A61K 31/496; A61P 25/28; A61P 37/02; A61P 43/00; C07D 311/30

(57) ABSTRACT

A dihydromyricetin (DHM) derivative and a preparation method and use thereof are provided. Different structures of the DHM derivative are provided to address the problem that DHM fails to maintain stable therapeutic efficacy in vivo due to poor inherent stability. The DHM derivative can be stably stored at a temperature of 25° C. to 45° C. and a pH of less than or equal to 9. The DHM derivative can regulate the immune function in the body, demonstrates a therapeutic effect for degenerative diseases, and can exert stable efficacy in vivo. Therefore, the DHM derivative is suitable for the drug development for humans, and has a promising application prospect.

### CROSS REFERENCE TO THE RELATED APPLICATIONS

This application is based upon and claims priority to Chinese Patent Application No. 202410891587.3, filed on Jul. 4, 2024, the entire contents of which are incorporated herein by reference.

### TECHNICAL FIELD

The present disclosure relates to the field of chemical synthesis, and specifically relates to a dihydromyricetin (DHM) derivative and a preparation method and uses thereof.

### BACKGROUND

DHM, also known as ampelopsin, was first isolated from the leaves of Fujian tea (i.e., Ampelopsis meliaefolia) in the Ampelopsis genus of the Vitaceae family by Kotake and Kubota in 1940. DHM is present in plants of the Vitaceae, Ericaceae, Burseraceae, Clusiaceae, and Salicaceae families, and is particularly abundant in Ampelopsis grossedentata (vine tea).

Current studies have shown that DHM possesses various effects, including anticancer, antioxidant, anti-inflammatory, antibacterial, hypotensive, hypoglycemic, hypolipidemic, and cardiovascular-protective effects. DHM can also provide protective effects and ameliorate the progression of diseases such as Alzheimer's disease (AD), brain aging, and muscle atrophy. DHM intrinsically possesses an extended π-conjugated system and strongly coordinating oxygen atoms. The optimal spatial structure of DHM enables the chelation of DHM with metal ions. DHM-metal complexes produced accordingly can be introduced into pharmaceuticals, foods, and cosmetics to exert antibacterial, antioxidant, and anti-aging effects. DHM includes six hydroxyl groups, and exhibits weak acidity. The solubility of DHM can be improved through esterification to enhance the antioxidant activity of DHM in fats. DHM esters produced accordingly can be incorporated into cosmetics to boost the oxidation resistance, prevent the skin aging, and reduce the addition of antimicrobial preservatives.

However, DHM exhibits poor stability and is prone to oxidation due to the presence of a pyrogallol-type vicinal trihydroxyl structure. The stability of DHM is affected by factors such as pH, temperature, and metal ions to some extent. Studies have shown that DHM remains stable at a pH of less than or equal to 4, but the increase of pH will lead to the acceleration of oxidation of DHM. When DHM is heated at a temperature of 100° C. or lower for 30 min or less, the chemical structure of DHM remains stable. However, as the temperature further increases, the stability of DHM deteriorates, and DHM may even undergo irreversible oxidation. Due to the inherent instability of DHM, DHM will lose its efficacy due to degradation during an administration process, which limits the medical use of DHM.

Therefore, the development of DHM derivatives with enhanced pharmacological and drug-like properties holds significant medical importance.

### SUMMARY

To address the problem that DHM fails to maintain stable therapeutic efficacy in vivo due to poor inherent stability, the present disclosure provides a DHM derivative. While retaining the pharmacological activity of DHM, the present disclosure can improve and modulate the physical and chemical properties of DHM, thereby enhancing the efficacy, safety, and stability.

Specifically, the present disclosure provides a DHM derivative and a pharmaceutically acceptable salt thereof, where the DHM derivative is a compound shown in a formula I:

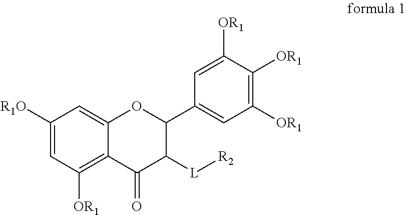

where R₁ is independently selected from C₁₋₆ alkyl, C₁₋₆ alkylcarbonyl, C₁₋₆ alkylsulfonyl, C₁₋₆ alkylsulfinyl, or H;
L is selected from —(CR₃R₄)n-C(═X)—, —C(═X)—(CR₃R₄)n-, —(CR₃R₄)m-, —(CR₃R₄)n-C(═O)—NH—, —(CR₃R₄)n-NH—C(═O)—, —C(═O)—NH—(CR₃R₄)n-, —NH—C(═O)—(CR₃R₄)n-, —(CR₃R₄)n-C(═O)—NH—(CR₃R₄)n-, —(CR₃R₄)n-NH—C(═O)—(CR₃R₄)n-, or O—C(═O)—(CR₃R₄)n-, where X is selected from O, S, and NH; n and m are each independently selected from integers of 0 to 6; and
R₃ and R₄ are each independently selected from H, C₁₋₆ alkyl, a halogen, hydroxyl, —NR₅R₆, or —CN;
R₂ is selected from 3- to 12-membered heterocyclyl that is optionally substituted with C₁₋₆ alkyl, a halogen, hydroxyl, —NR₅R₆, —CN, —SF₅, or C₁₋₆ alkoxy; or
R₂ is selected from —NR₅R₆,
where R₅ and R₆ are each independently selected from H, C₁₋₆ alkyl, and —C(═NH)NH₂.

As a preferred embodiment, R₁ is independently selected from C₁₋₆ alkyl or H. As a preferred embodiment, L is selected from —(CR₃R₄)m-, where R₃ and R₄ are each independently selected from H and C₁₋₆ alkyl.

As a preferred embodiment, R₂ is selected from 5- to 10-membered heterocyclyl that is optionally substituted with C₁₋₆ alkyl, a halogen, hydroxyl, —NR₅R₆, —CN, or C₁₋₆ alkoxy.

As a preferred embodiment, R₂ is selected from —NR₅R₆, where R₅ and R₆ are each independently selected from H, C₁₋₆ alkyl, and —C(═NH)NH₂.

As a further preferred embodiment, the C₁₋₆ alkyl is selected from methyl, ethyl, n-propyl, isopropyl, n-butyl, isobutyl, tert-butyl, n-pentyl, isopentyl, neopentyl, or n-hexyl.

As a further preferred embodiment, the heterocyclyl is selected from azacyclobutyl, oxacyclobutyl, tetrahydrofuranyl, dioxolyl, pyrrolidinyl, imidazolidinyl, pyrazolidinyl, pyrrolinyl, tetrahydropyranyl, piperidinyl, morpholinyl, dithianyl, thiomorpholinyl, piperazinyl, trithianyl, or diazepanyl.

Furthermore, the heterocyclyl is selected from tetrahydrofuranyl, pyrrolidinyl, tetrahydropyranyl, piperidinyl, morpholinyl, or piperazinyl.

According to an embodiment of the present disclosure, the compound shown in the formula I is selected from the following structures:

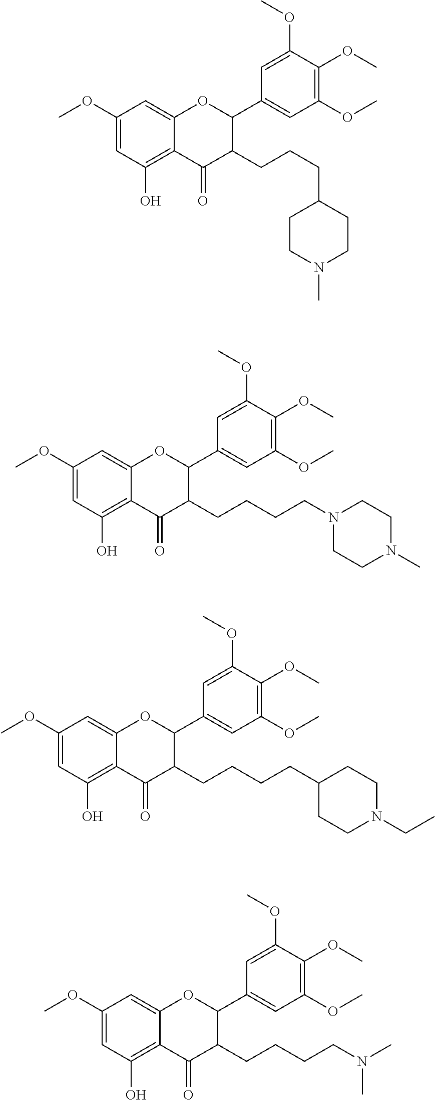

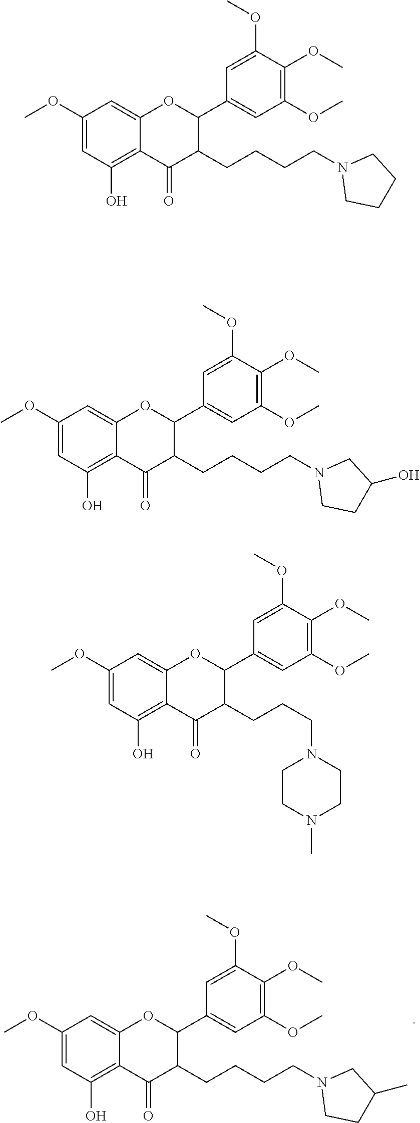

Another objective of the present disclosure is to provide a preparation method of the compound shown in the formula I, including the following step:

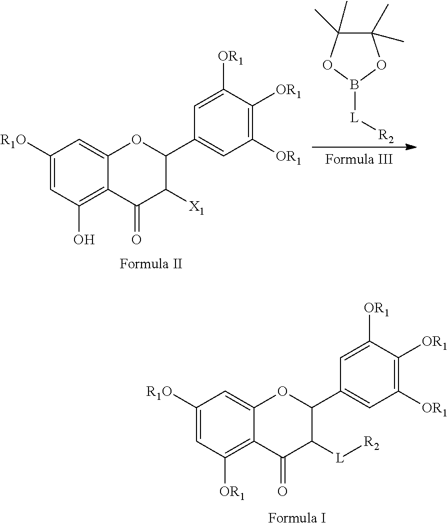

allowing a compound shown in a formula II to react with a compound shown in a formula III to produce the compound shown in the formula I, where X1 is selected from Br, I, or OTf.

The present disclosure also provides a pharmaceutical composition including a compound of formula I, or a pharmaceutically acceptable salt thereof, formulated for administration via a route selected from oral administration, injection, rectal administration, nasal administration, pulmonary administration, topical administration, buccal and sublingual administration, vaginal administration, parenteral administration, subcutaneous administration, intramuscular administration, intravenous administration, intradermal administration, intrathecal administration, and epidural administration.

The pharmaceutical composition is preferably administered orally. A dosage form for the oral administration is not specifically limited, and any oral dosage form well known in the art may be adopted, preferably including a tablet, a capsule, a suspension, or an oral dosage form known in the art such as an oral solution. As an oral dosage form, the pharmaceutical composition is administered at a dose of 500 mg/d to 1,500 mg/d, for example, which is preferably 700 mg/d to 1,200 mg/d, more preferably 800 mg/d to 1,000 mg/d, and most preferably 1,000 mg/d.

A medication duration of the pharmaceutical composition of the present disclosure may be determined according to the severity of a disease, and is preferably at least 1 month, such as 1, 2, 3, 4, 5, or 6 months. The pharmaceutical composition may be taken for a lifetime due to medical needs.

According to an embodiment of the present disclosure, the pharmaceutical composition further includes a pharmaceutically acceptable adjuvant. Preferably, the adjuvant is selected from at least one of adjuvants including, but not limited to, a filler, a disintegrant, a binder, a lubricant, a surfactant, a corrigent, a wetting agent, a pH regulator, a solubilizer or cosolvent, or an osmotic pressure regulator. Those skilled in the art can easily determine how to select an appropriate adjuvant and a corresponding amount thereof based on a specific dosage form.

According to an embodiment of the present disclosure, the pharmaceutical composition may further include one or more additional therapeutic agents.

The present disclosure also provides a use of at least one of the compound shown in the formula I or the pharmaceutically acceptable salt thereof, and the pharmaceutical composition in preparation of a drug. The drug can be used for treating insomnia, a sleep disorder, anxiety, cognitive decline, memory impairment, and a neurodegenerative disease. The neurodegenerative disease includes neuroinflammation, AD, Parkinson's disease, Huntington's disease, or amyotrophic lateral sclerosis.

The present disclosure also provides a use of at least one of the compound shown in the formula I or the pharmaceutically acceptable salt thereof, and the pharmaceutical composition in preparation of a drug, where the use includes a use in treating an immune system disorder. The immune system disorder includes rheumatoid arthritis, muscular dystrophy, systemic lupus erythematosus, spinal cord syndrome, multiple sclerosis, systemic sclerosis, scleroderma, small cell lung cancer syndrome, renal tuberculosis, lymphoma, hepatitis B, and chronic lymphocytic leukemia.

The present disclosure also provides a use of at least one of the compound shown in the formula I or the pharmaceutically acceptable salt thereof, and the pharmaceutical composition in preparation of a drug, where the use includes a use in delaying/resisting aging.

### Beneficial Effects

(1) The DHM derivative of the present disclosure can be stably stored at a temperature of 25° C. to 45° C. and a pH of less than or equal to 9, and is suitable for the drug development for humans.
(2) The DHM derivative of the present disclosure can regulate the immune function in the body with a significantly stronger effect than DHM.
(3) The DHM derivative of the present disclosure demonstrates a therapeutic effect for degenerative diseases, and can exert stable efficacy in vivo.

### BRIEF DESCRIPTION OF THE DRAWINGS

FIG. 1 shows the detection results of Aβ1-42 in rat brains;

FIG. 2 shows the expression of Beclin1 in rat brains;

FIG. 3 shows the expression of LC3-II/LC3-I in rat brains;

FIG. 4 shows the expression of p62 in rat brains;

FIG. 5 shows the expression of LAMP1 in rat brains;

FIG. 6 shows the expression of Cathepsin D in rat brains.

### DETAILED DESCRIPTION OF THE EMBODIMENTS

### Definitions and Explanations of Terms

Unless otherwise specified, the definitions for groups and terms mentioned in the specification and claims of the present application, including definitions provided as examples, exemplary definitions, preferred definitions, definitions listed in tables, and definitions for the specific compounds in embodiments, may be combined and associated with each other in any way. Group definitions and compound structures resulting from such combination and association shall be construed as falling within the scope defined in the description and/or claims of the present application.

Unless otherwise stated, a numerical range recorded in the specification and claims means that each specific integer value in the numerical range is disclosed at least. For example, a numerical range of 0 to 6 shall be construed as disclosing each integer value within the numerical range of 0 to 6, namely 0, 1, 2, 3, 4, 5, and 6. When a numerical value of 0 is taken, m=0 represents a chemical bond in —(CR₃R₄)m-, for example.

The term “halogen” represents fluorine, chlorine, bromine, and iodine. In other words, F, Cl, Br, and I may be described as “halogen” in this specification.

The phrase “optionally substituted with a substituent” encompasses both the case where there is no substitution and the case there is substitution with one or more substituents. For example, “optionally substituted with one, two, or more R groups” means that there is no substitution (non-substitution) with R or there is substitution with one, two, or more R groups.

The term “alkyl” refers to linear or branched hydrocarbon group that consists solely of carbon and hydrogen atoms, free of unsaturated bonds, and has, for example, 1 to 6 carbon atoms that are connected to the remaining moiety of the molecule through single bonds. Examples of the alkyl include, but are not limited to, methyl, ethyl, n-propyl, isopropyl, n-butyl, isobutyl, tert-butyl, pentyl, isopentyl, neopentyl, and hexyl. The alkyl may be unsubstituted or may be substituted with one or more suitable substituents. The alkyl group can also be an isotopologue of a naturally abundant alkyl group enriched in carbon and/or hydrogen isotopes (i.e., deuterium or tritium).

The term “C₁₋₆ alkyl”, when used alone or as a part of another substituent, shall be construed as indicating linear or branched saturated monovalent hydrocarbyl with 1, 2, 3, 4, 5, or 6 carbon atoms. The alkyl is, for example, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, etc., or an isomer thereof.

The term “3- to 12-membered heterocyclyl” refers to a saturated or unsaturated non-aromatic ring or ring system, such as a 4-, 5-, 6-, or 7-membered monocyclic ring, a 7-, 8-, 9-, 10-, 11-, or 12-membered bicyclic ring (such as a fused ring, a bridged ring, and a spiro-ring), or a tricyclic ring. Moreover, the heterocyclyl includes at least one, such as 1, 2, 3, 4, 5, or more, heteroatom selected from O, S, and N, where N and S may optionally be oxidized into various oxidation states, such as nitrogen oxides, —S(O)—, or —S(O)₂—. Preferably, the heterocyclyl may be selected from “3- to 10-membered heterocyclyl”. The term “3- to 10-membered heterocyclyl” refers to a saturated or unsaturated non-aromatic ring or ring system including at least one heteroatom selected from O, S, and N. The heterocyclyl may be attached to the remaining moiety of the molecule through any one of the carbon atoms or a nitrogen atom (if present). The heterocyclyl may include a fused ring or a bridged ring or a spiro-ring. In particular, the heterocyclyl may include, but is not limited to, a 4-membered ring, such as azacyclobutyl or oxacyclobutyl; a 5-membered ring, such as tetrahydrofuranyl, dioxolyl, pyrrolidinyl, imidazolidinyl, pyrazolidinyl, pyrrolinyl; or a 6-membered ring, such as tetrahydropyranyl, piperidinyl, morpholinyl, dithianyl, thiomorpholinyl, piperazinyl, or trithianyl; or a 7-membered ring, such as diazepanyl. Optionally, the heterocyclyl can be benzo-fused. The heterocyclyl may be bicyclic, including, but not limited to, a 5,5-membered ring, such as a hexahydrocyclopenta[c]pyrrol-2(1H)-yl ring, or a 5,6-membered bicyclic ring, such as a hexahydropyrrolo[1,2-a]pyrazin-2(1H)-yl ring. The heterocyclyl may be partially unsaturated. That is, the heterocyclyl may include one or more double bonds, including, but not limited to, dihydrofuranyl, dihydropyranyl, 2,5-dihydro-1H-pyrrolyl, 4H-[1,3,4]thiadiazinyl, 1,2,3,5-tetrahydrooxazolyl, or 4H-[1,4]thiazinyl. Alternatively, the heterocyclyl may be benzo-fused, including, but not limited to, dihydroisoquinolinyl. When the 3- to 12-membered heterocyclyl is linked to other groups to produce the compound of the present disclosure, the linking may refer to linking of a carbon atom of the 3- to 12-membered heterocyclyl or a heterocyclic atom of a ring of the 3- to 12-membered heterocyclyl to other groups. For example, when the 3- to 12-membered heterocyclyl is selected from piperazinyl, the linking may refer to linking of a nitrogen atom of piperazinyl to other groups. Alternatively, when the 3- to 12-membered heterocyclyl is selected from piperidinyl, the linkage may be through the ring nitrogen atom and/or the carbon atom para to said nitrogen atom.

In the present application, the term “salt” or “pharmaceutically acceptable salt” includes a pharmaceutically acceptable acid-addition salt and a pharmaceutically acceptable base-addition salt. The term “pharmaceutically acceptable” means that compounds, materials, compositions, and/or dosage forms are suitable for the use in contact with human and animal tissues in a reliable medical determination range, which does not have excessive toxicity, irritability, anaphylaxis, or other problems or complications and is commensurate with a reasonable benefit/risk ratio.

The phrases such as “one embodiment” and “an embodiment” mentioned in the specification indicate that an embodiment described accordingly may include a particular aspect, feature, structure, component, or characteristic, but not every embodiment necessarily includes the aspect, feature, structure, component, or characteristic. Moreover, such a phrase may, but does not necessarily, refer to the same embodiment as mentioned elsewhere in the specification. When a specific aspect, feature, structure, element, or characteristic is described in connection with an embodiment, whether explicitly described or not, it is within the knowledge of those skilled in the art to affect or associate the aspect, feature, structure, element, or characteristic with other embodiments.

As understood by those skilled in the art, all numerical values, including those indicating amounts and properties such as molecular weight and reaction conditions for components, are all approximate values, and should be considered as being optionally modified by the term “about” in all cases. These values may vary depending on the desired properties sought by those skilled in the art using the teachings described herein. It should also be understood that these values inherently encompass variability which necessarily arises from a standard deviation found in a respective test measurement.

The term “effective amount” refers to an amount sufficient to treat a disease, disorder, and/or condition or to produce a desired effect. For example, an effective amount may be an amount sufficient to alleviate the progression or severity of a condition or symptom being treated.

The determination of a therapeutically effective amount is well within the capability of those skilled in the art. The effective amount includes an amount of the compound described herein or an amount of the composition described herein that is sufficient to treat or prevent a disease or disorder in a subject or control symptoms of the disease or disorder, for example. Thus, the effective amount generally refers to an amount for providing a desired effect.

Alternatively, the term “effective dose” or “therapeutically effective dose” as used herein refers to an amount of a preparation or composition that is sufficient to alleviate one or more symptoms of a disease or condition being treated to some extent. An outcome may involve the reduction and/or alleviation of a sign, a symptom, or a cause of a disease, or any other desired alteration in a biological system. For example, an “effective amount” for a therapeutic use refers to an amount of a composition including the compound for clinically alleviating a symptom of a disease significantly disclosed herein. The appropriate “effective amount” in any individual case may be determined using a technique such as dose-escalation study. A dose may involve a single administration or multiple administrations. However, the accurate determination of the effective dose may be based on individual factors of a patient, including, but not limited to: an age and a body type of the patient, a type, a severity level, and a stage of a disease, an administration route of the composition, a type or an extent of an adjuvant therapy, an ongoing disease course, and a required therapy type (such as aggressive therapy and conventional therapy).

The term “treatment” includes (i) prevention of the occurrence of a disease, pathology, or a medical condition (prophylaxis); (ii) suppression of a disease, pathology, or a medical condition or arrest of the progression of the disease, the pathology, or the medical condition; (iii) amelioration of a disease, pathology, or a medical condition; and/or (iv) alleviation of symptoms associated with a disease, pathology, or a medical condition. Thus, the term “treatment” may extend to prevention, and may include preventing, protecting against, reducing, arresting, or reversing the progression or severity of a condition or symptom being treated. Therefore, the term “treatment” may, where appropriate, include pharmaceutical, therapeutic, nutritional, and/or prophylactic administration.

As used herein, the term “subject” or “patient” refers to an individual that undergoes symptoms of or is at risk of a disease or other malignant tumors. The term “patient” may refer to a human or non-human subject, and may include an animal strain or species serving as a “model system” for a research purpose, such as the mouse model described herein. Further, the patient may refer to any living organism or may include adults or juveniles (such as children) and preferably mammals (such as human or non-human mammals), and can benefit from the administration of the composition contemplated herein. Examples of the mammals include, but are not limited to, any member of the mammalian family: humans; non-human primates, such as chimpanzees and other apes and monkey species; farm animals, such as cattle, horses, sheep, goats, and swine; domestic animals, such as rabbits, dogs, and cats; and laboratory animals, including rodents such as rats, mice, and swine. Examples of the non-mammals include, but are not limited to, birds and fish. In an embodiment for the method provided herein, the mammals are humans. As used herein, the terms “applying” and “administering” can be used interchangeably, and refer to delivering the compound disclosed into a subject through a method or route for at least partially locating the compound at a desired site. The compound may be administered through any appropriate route such that the compound can be delivered to a desired site in a subject.

The compound and composition described herein may be administered together with other compositions to enhance the stability and activity of the composition, or can be administered in combination with other supplements, nutritions, therapeutic agents, or drugs.

The term “inhibition” refers to the mitigation, halting, or reversion of growth or progression of a disease, an infection, a condition, or a cell population. For example, the inhibition may be greater than approximately 20%, 40%, 60%, 80%, 90%, 95%, or 99% compared to the growth or progression occurring when there is no treatment or exposure.

In the present disclosure, whenever the term “include” is used, the terms “consist of” and “comprise or consist essentially of” are encompassed as optional variants. As used herein, the term “comprise” and the term “include” or “characterized by comprising” are synonymous, which are inclusive or open-ended, and do not exclude additional and unrecited elements or method steps. As used herein, the term “consisting of” excludes any element, step, or ingredient not specified in the defined aspect. As used herein, the term “consisting essentially of” does not exclude a material or step that does not substantially affect the basic and novel features of the defined aspect. In each instance herein, any one of the terms “include”, “consisting essentially of”, and “consisting of” may be replaced with either of the other two terms. The disclosure described illustratively herein may be implemented appropriately without limitation or restriction by any one or more elements not specifically disclosed herein.

The following examples are intended to illustrate the present disclosure and should not be construed as limiting the scope of the present disclosure. Those skilled in the art will readily understand that numerous other ways to implement the present disclosure are provided in the examples. It should be understood that numerous variations and modifications may be made within the scope of the present disclosure.

### Example 1

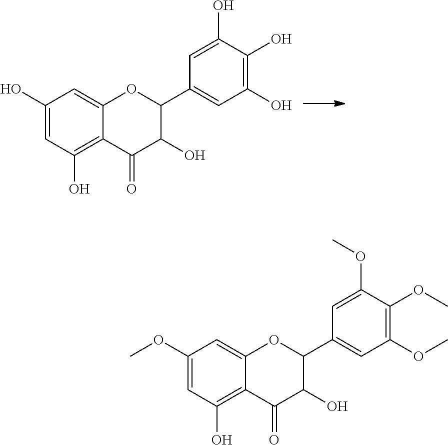

In a N₂ atmosphere, 200 mg (0.62 mmol) of DHM was dissolved in 7 mL of anhydrous N,N-dimethylformamide (DMF). 552 mg (2.5 mmol) of anhydrous K₂CO₃ was added, and 383 mg (2.7 mmol) of iodomethane was added slowly. A reaction was conducted at 40° C. for 24 h. 1 mL of acetic acid was added, the mixture was filtered, the solvent was recovered by evaporation under reduced pressure, and the residue was purified by silica gel column chromatography (V_{petroleum ether}:V_{ethyl acetate}=6:1) to produce a 7,3′,4′,5′-tetramethoxydihydromyricetin intermediate, with a yield of 26.2%.

### Example 2

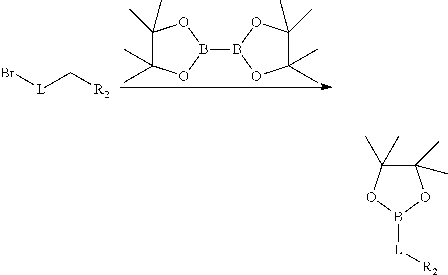

To a vial containing a degassed (3×vacuum/Ar) mixture of Br-L-R2 (4.11 mmol), bis(pinacolato)diboron (1.25 g, 4.94 mmol), and potassium acetate (1.21 g, 12.3 mmol) in dioxane (10 mL) was added PdCl₂(dppf) CH₂Cl₂ adduct (0.090 g, 0.123 mmol). A resulting reaction mixture was degassed, sealed, heated at 110° C. for 16 h, diluted with water, and then subjected to extraction with EtOAc. A resulting organic phase was concentrated and subjected to preliminary purification by flash chromatography (EtOAc/hexane) to produce intermediates with structures shown in the table below.

TABLE 1
Intermediates L2-1 to L2-8

| | | | |
|---|---|---|---|
| L2-1 | 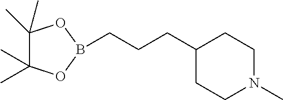 | L2-5 | 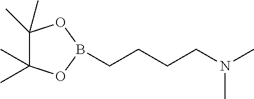 |
| | HRMS (m/z): [M + H]⁺ 266.3431 | | HRMS (m/z): [M + H]⁺ 226.2138 |
| L2-2 | 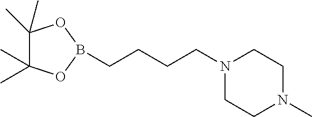 | L2-6 | 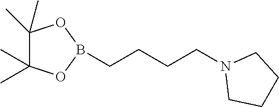 [SMILES: CC1(C)OB(CCCCN2CCCC2)OC1(C)C] |
| | HRMS (m/z): [M + H]⁺ 281.2539 | | HRMS (m/z): [M + H]⁺ 252.2247 |
| L2-3 | 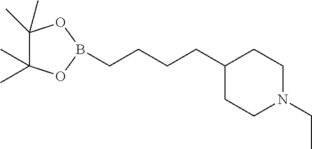 [SMILES: CCN1CCC(CCCCB2OC(C)(C)C(C)(C)O2)CC1] | L2-7 | 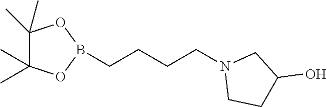 [SMILES: CC1(C)OB(CCCCN2CCC(O)C2)OC1(C)C] |
| | HRMS (m/z): [M + H]⁺ 294.2741 | | HRMS (m/z): [M + H]⁺ 268.2252 |
| L2-4 | 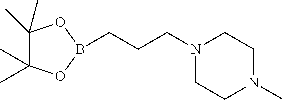 | L2-8 | 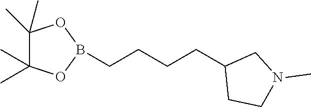 |
| | HRMS (m/z): [M + H]⁺ 267.2472 | | HRMS (m/z): [M + H]⁺ 266.2442 |

### Example 3

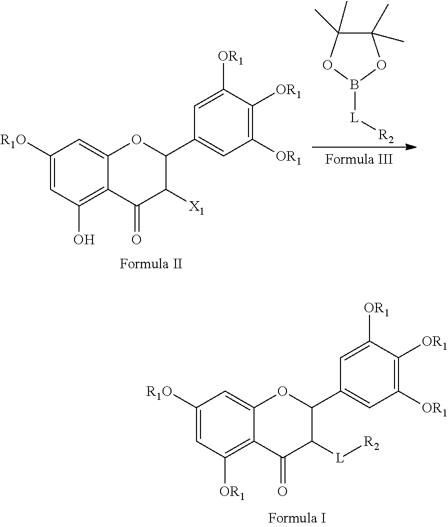

At room temperature, a compound of a formula II (0.3 mmol) was dissolved in 1,4-dioxane (2 mL) and water (0.4 mL), and then a compound of a formula III (L2-1 to L2-8, 1.05 mmol), cesium carbonate (1.07 mmol), and Pd(PPH₃)₄ (0.036 mmol) were added successively. A resulting reaction system was stirred overnight at 95° C. in a nitrogen atmosphere. After a reaction was completed, a resulting reaction solution was cooled to room temperature, diluted with water (20 mL), and then subjected to extraction with ethyl acetate (3×20 mL). Resulting organic phases were combined, washed with a saturated sodium chloride solution (40 mL), dried with anhydrous sodium sulfate, and filtered. A resulting filtrate was concentrated under reduced pressure to produce a crude product. The crude product was purified by preparative thin layer chromatography (mobile phase:ethyl acetate/petroleum ether=1:2) to produce yellow oily compounds L3-1 to L3-8 with structures shown in Table 2.

TABLE 2
DHM derivatives L3-1 to L3-8

| | | | |
|---|---|---|---|
| L3-1 | 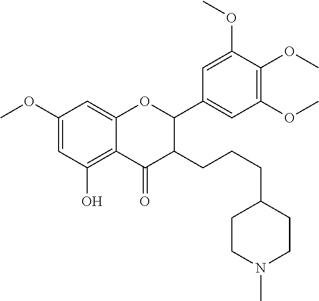 | L3-5 | 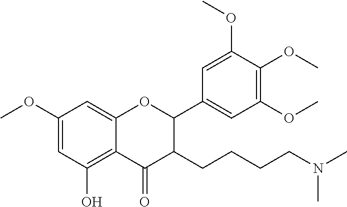 |
| | HRMS (m/z): [M + H]⁺ 500.2653 | | HRMS (m/z): [M + H]⁺ 460.2386 |
| L3-2 | 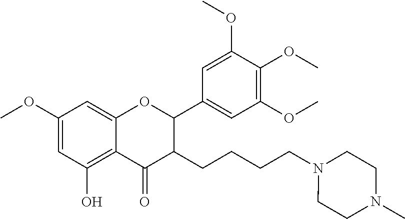 | L3-6 | 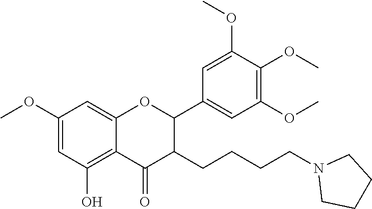 [SMILES: COc1cc(O)c2c(c1)OC(c1cc(OC)c(OC)c(OC)c1)C(CCCCN1CCCC1)C2=O] |
| | HRMS (m/z): [M + H]⁺ 515.2731 | | HRMS (m/z): [M + H]⁺ 486.2453 |
| L3-3 | 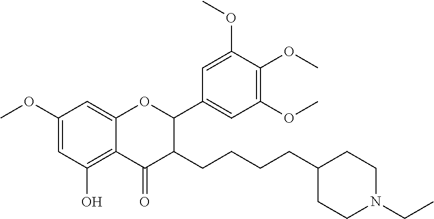 [SMILES: CCN1CCC(CCCCC2C(=O)c3c(O)cc(OC)cc3OC2c2cc(OC)c(OC)c(OC)c2)CC1] | L3-7 | 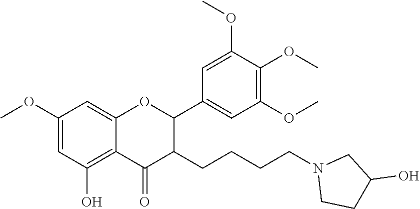 [SMILES: COc1cc(O)c2c(c1)OC(c1cc(OC)c(OC)c(OC)c1)C(CCCCN1CCC(O)C1)C2=O] |
| | HRMS (m/z): [M + H]⁺ 528.2954 | | HRMS (m/z): [M + H]⁺ 502.2475 |
| L3-4 | 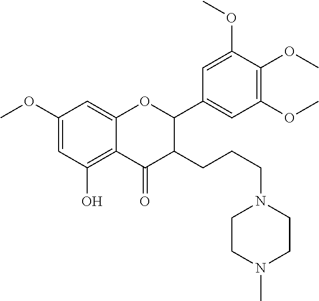 | L3-8 | 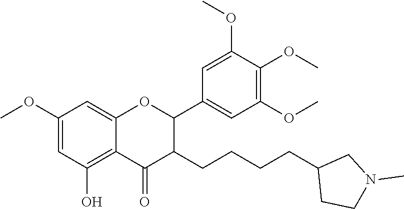 |
| | HRMS (m/z): [M + H]⁺ 501.2642 | | HRMS (m/z): [M + H]⁺ 500.2671 |

### Example 4 Stability Test

In this example, the stability of DHM and the derivatives of the present disclosure under different temperatures and light conditions were investigated.

A test method was as follows:

Study on the stability of DHM and derivatives thereof at different temperatures: 1 mL of a 1 mg/mL DHM or derivative solution (dissolved in 60% absolute ethanol) was mixed with 9 mL of a phosphate buffer (PB) (0.2 M, pH 8.0). A resulting mixed solution was placed in water baths at different temperatures (25° C., 37° C., and 45° C.), and sealed and exposed to natural scattered light for 1 h\2 h\4 h. A residual amount was determined by high-performance liquid chromatography (HPLC).

Influence of different pH values: 1 mL of a 1 mg/mL DHM or derivative solution (dissolved in 60% absolute ethanol) was mixed with 9 mL of each of PBs at varying pH values. A resulting mixed solution was placed at room temperature (25° C.) for 8 h. Then, a sample was collected and tested by HPLC to determine a residual amount of DHM.

HPLC analysis conditions: Agilent 1260 HPLC system equipped with a photodiode array detector, an automatic sampler, and a symmetrical C18 chromatographic column (4.6 mm×250 mm, 5.0 μm; Waters, USA); mobile phase: 25% of acetonitrile and 75% of an aqueous phase (including 0.1% of glacial acetic acid) for isocratic elution; flow rate: 1 mL/min; column temperature: 40° C.; detection wavelength: 291 nm; and injection volume for the stability study: 10 μL.

TABLE 3
Temperature stability study

| Compo- | 25° C. | | | 37° C. | | | 45° C. | | |
|---|---|---|---|---|---|---|---|---|---|
| nent | 1 h | 2 h | 4 h | 1 h | 2 h | 4 h | 1 h | 2 h | 4 h |
| DHM | 50% | 25% | 7% | 34% | 14% | 5% | 23% | 10% | 2% |
| L3-1 | 100% | 96% | 94% | 100% | 96% | 92% | 96% | 88% | 85% |
| L3-2 | 100% | 97% | 95% | 100% | 97% | 92% | 97% | 89% | 83% |
| L3-3 | 100% | 97% | 96% | 100% | 97% | 93% | 97% | 90% | 84% |
| L3-4 | 100% | 96% | 94% | 100% | 96% | 91% | 96% | 87% | 82% |
| L3-5 | 100% | 98% | 96% | 100% | 98% | 92% | 98% | 88% | 82% |
| L3-6 | 100% | 97% | 95% | 100% | 97% | 93% | 97% | 87% | 83% |
| L3-7 | 100% | 97% | 94% | 100% | 97% | 94% | 97% | 91% | 84% |
| L3-8 | 100% | 96% | 95% | 100% | 96% | 93% | 96% | 90% | 85% |

It can be seen that, after being stored at room temperature for 4 h or more and placed in an environment with a temperature of 37° C. or higher, DHM undergoes oxidation spontaneously to produce other components. Therefore, if administered to the human body, DHM will quickly lose its pharmacological activity. However, the derivatives of the present disclosure are less affected by a temperature, and have a residual amount remaining almost unchanged. Therefore, the derivatives of the present disclosure are suitable for drug development.

TABLE 4
pH stability study

| Component | pH 3 | pH 4 | pH 5 | pH 6 | pH 7 | pH 8 | pH 9 |
|---|---|---|---|---|---|---|---|
| DHM | 100% | 98% | 97% | 90% | 50% | 0% | 0% |
| L3-1 | 100% | 100% | 100% | 100% | 98% | 94% | 89% |
| L3-2 | 100% | 100% | 100% | 100% | 99% | 95% | 90% |
| L3-3 | 100% | 100% | 100% | 100% | 98% | 96% | 92% |
| L3-4 | 100% | 100% | 100% | 100% | 97% | 94% | 90% |
| L3-5 | 100% | 100% | 100% | 100% | 98% | 96% | 92% |
| L3-6 | 100% | 100% | 100% | 100% | 97% | 95% | 92% |
| L3-7 | 100% | 100% | 100% | 100% | 96% | 94% | 91% |
| L3-8 | 100% | 100% | 100% | 100% | 98% | 95% | 90% |

Under acidic conditions, both DHM and the derivatives of the present disclosure can exist stably. Under neutral conditions, DHM undergoes structural changes and cannot exist stably. The derivatives of the present disclosure can also exist stably under neutral conditions, which makes administration modes such as injection and oral administration possible. Under alkaline conditions, DHM undergoes complete structural changes. The DHM component can no longer be detected in a DHM sample stored in an alkaline solution. Under alkaline conditions, the derivatives of the present disclosure can still exist relatively stably. Many parts of the human body tend to be under neutral to mildly alkaline conditions. As a result, the derivatives of the present disclosure can be used in various scenarios. An 8 h treatment period is adopted to simulate the conditions of a drug in the human body because 8 h is sufficient for the human body to complete absorption.

### Example 5 Studies on Immunomodulation

a. A total of 110 SPF-grade male mice were selected and grouped according to the table below: group 1: a blank group (normal mice), and groups 2 to 11: model mice constructed by intraperitoneally injecting mice with cyclophosphamide at 80 mg/kg/d consecutively for 3 d. During the modeling, mice in the blank group were each intraperitoneally injected with an equal volume of normal saline consecutively for 3 d. After the modeling, in the groups 3 to 11, mice were each injected with DHM and derivatives 1 to 8 at 2 mg/kg/day, respectively, and in the groups 1 and 2, mice were each intraperitoneally injected with an equal amount of normal saline. Each group was injected once a day consecutively for 17 d. After the intraperitoneal injection was completed, mice were each fasted for 24 h, and measured for a body mass by an electronic balance. Eyeballs were removed, blood was collected, and then mice each were sacrificed through cervical dislocation. Spleen and thymus tissues were collected and stored in a −80° C. freezer for later use.

b. An immune organ index was determined by the weighing method. The spleen and thymus tissues were subjected to connective and adipose tissue removal, rinsed with normal saline, suck-dried with a filter paper, and weighed with a semi-micro analytical balance (immune organ masses). A thymus index and a spleen index were calculated. Immune organ index=immune organ mass (mg)/body mass (g).

TABLE 4
Immune organ indexes

| Group | Mouse | Intraperitonealinjection | Numberofmice | Spleenindex | Thymusindex |
|---|---|---|---|---|---|
| 1 | Normal mice | Normal saline | 10 | 3.87 ± 0.29 | 3.52 ± 0.20 |
| 2 | Model mice | Normal saline | 10 | 2.36 ± 0.21 | 2.40 ± 0.88 |
| 3 | Model mice | L3-1 | 10 | 3.62 ± 0.31 | 3.33 ± 0.31 |
| 4 | Model mice | L3-2 | 10 | 3.73 ± 0.28 | 3.44 ± 0.43 |
| 5 | Model mice | L3-3 | 10 | 3.59 ± 0.26 | 3.30 ± 0.35 |
| 6 | Model mice | L3-4 | 10 | 3.66 ± 0.34 | 3.37 ± 0.41 |
| 7 | Model mice | L3-5 | 10 | 3.63 ± 0.25 | 3.32 ± 0.39 |
| 8 | Model mice | L3-6 | 10 | 3.72 ± 0.33 | 3.41 ± 0.56 |
| 9 | Model mice | L3-7 | 10 | 3.61 ± 0.24 | 3.28 ± 0.32 |
| 10 | Model mice | L3-8 | 10 | 3.68 ± 0.27 | 3.35 ± 0.45 |
| 11 | Model mice | DHM | 10 | 3.07 ± 0.32 | 2.88 ± 0.37 |

According to the results in the table above, compared to the group 2, model mice in the groups 3 to 11 have a spleen index and a thymus index that are both improved, and the DHM derivatives lead to a significantly better improvement effect than the DHM. It indicates that both the DHM and the DHM derivatives can enhance the immune function in the body. However, in contrast to the DHM, the DHM derivatives of the present disclosure can effectively counteract the immunosuppression induced by cyclophosphamide, and lead to enhanced effects.

c. A mouse spleen from each group was weighed and ground, and spleen single cells were isolated using a lymphocyte separation medium (mouse spleen lymphocyte separation kit, Tianjin Hao Yang Biological Products Technology Co., Ltd.). Based on a cell counting result, a cell suspension with a concentration of 1×10⁴ cells/μL was prepared. 50 μL of a single-cell suspension prepared in advance was taken, and 1 μL of each of anti-mouse CD3⁺, CD4⁺, and CD8⁺ fluorescent antibodies was added. Incubation was conducted at a constant temperature of 4° C. for 30 min in the dark. Cells were then washed once with phosphate buffered saline (PBS), and centrifuged at 2,000 rpm for 5 min. A resulting supernatant was discarded. 500 μL of PBS was added to a resulting pellet, and thorough mixing was conducted gently. Each cell subset proportion was then detected by a flow cytometer (Accui C5, BD Biosciences, USA).

Results were as follows:

TABLE 5
T lymphocyte subset indexes

| | | Intraperitoneal | Number | | | | |
|---|---|---|---|---|---|---|---|
| Group | Mouse | injection | of mice | CD3⁺/% | CD4⁺/% | CD8⁺/% | CD4⁺/CD8⁺ |
| 1 | Normal | Normal saline | 10 | 44.23 ± 1.75 | 34.18 ± 4.52 | 23.62 ± 3.84 | 1.45 ± 0.15 |
| | mice | | | | | | |
| 2 | Model | Normal saline | 10 | 32.84 ± 2.38 | 23.59 ± 3.75 | 19.16 ± 2.32 | 1.23 ± 0.12 |
| | mice | | | | | | |
| 3 | Model | L3-1 | 10 | 40.55 ± 1.83 | 31.57 ± 4.35 | 22.68 ± 1.92 | 1.39 ± 0.19 |
| | mice | | | | | | |
| 4 | Model | L3-2 | 10 | 42.33 ± 2.21 | 32.46 ± 3.85 | 22.75 ± 1.67 | 1.43 ± 0.12 |
| | mice | | | | | | |
| 5 | Model | L3-3 | 10 | 41.55 ± 1.59 | 32.64 ± 4.57 | 21.92 ± 1.94 | 1.44 ± 0.13 |
| | mice | | | | | | |
| 6 | Model | L3-4 | 10 | 41.58 ± 2.36 | 31.34 ± 3.95 | 22.31 ± 1.69 | 1.40 ± 0.14 |
| | mice | | | | | | |
| 7 | Model | L3-5 | 10 | 41.26 ± 2.69 | 31.33 ± 4.08 | 22.27 ± 1.22 | 1.41 ± 0.16 |
| | mice | | | | | | |
| 8 | Model | L3-6 | 10 | 42.88 ± 1.95 | 32.54 ± 2.76 | 22.95 ± 1.05 | 1.42 ± 0.17 |
| | mice | | | | | | |
| 9 | Model | L3-7 | 10 | 41.79 ± 2.35 | 31.31 ± 4.55 | 22.51 ± 2.54 | 1.39 ± 0.18 |
| | mice | | | | | | |
| 10 | Model | L3-8 | 10 | 42.97 ± 2.69 | 32.04 ± 2.58 | 22.75 ± 1.83 | 1.31 ± 0.18 |
| | mice | | | | | | |
| 11 | Model | DHM | 10 | 37.32 ± 2.77 | 27.63 ± 3.24 | 21.03 ± 2.13 | 1.31 ± 0.13 |
| | mice | | | | | | |

According to the above results, model mice of the groups 3 to 11 have larger CD³⁺, CD⁴⁺, and CD⁸⁺ T lymphocyte subset proportions and a larger CD⁴⁺/CD⁸⁺ ratio in spleens than model mice of the group 2. It indicates that the DHM and the DHM derivatives of the present disclosure can significantly improve the T lymphocyte subset indexes in spleens of immunosuppressed model mice, and the DHM derivatives demonstrate a more significant improvement effect than the DHM.

d. Biological indexes were detected. On day 17 of the test, eyeballs were removed from mice in each group, and blood was collected, allowed to stand for 2 h to 3 h, and centrifuged at 3,500 r/min for 15 min. A resulting serum in an upper layer was collected and tested for TNF-α, IL-6, and IL-8 levels by enzyme-linked immunosorbent assay (ELISA) (a test kit was purchased from Shanghai Enzyme-linked Biotechnology Co., Ltd.).

TABLE 6
TNF-α, IL-6, and IL-8 levels in mouse serum

| | | Intraperitoneal | Number | | | |
|---|---|---|---|---|---|---|
| Group | Mouse | injection | of mice | IL-6(pg/mL) | IL-8(pg/mL) | TNF-α(pg/mL) |
| 1 | Normal | Normal saline | 10 | 62.88 ± 7.55 | 41.69 ± 11.22 | 293.58 ± 54.18 |
| | mice | | | | | |
| 2 | Model | Normal saline | 10 | 96.25 ± 12.76 | 88.57 ± 15.98 | 445.28 ± 57.96 |
| | mice | | | | | |
| 3 | Model | L3-1 | 10 | 71.58 ± 8.94 | 49.26 ± 14.08 | 335.27 ± 44.89 |
| | mice | | | | | |
| 4 | Model | L3-2 | 10 | 66.43 ± 10.22 | 46.08 ± 12.61 | 320.47 ± 58.76 |
| | mice | | | | | |
| 5 | Model | L3-3 | 10 | 70.94 ± 9.14 | 51.25 ± 14.05 | 330.75 ± 53.21 |
| | mice | | | | | |
| 6 | Model | L3-4 | 10 | 68.97 ± 12.05 | 48.62 ± 13.54 | 319.45 ± 53.34 |
| | mice | | | | | |
| 7 | Model | L3-5 | 10 | 67.46 ± 14.15 | 47.54 ± 12.59 | 323.58 ± 60.44 |
| | mice | | | | | |
| 8 | Model | L3-6 | 10 | 65.87 ± 14.51 | 44.98 ± 10.80 | 316.25 ± 64.68 |
| | mice | | | | | |
| 9 | Model | L3-7 | 10 | 70.01 ± 15.26 | 50.27 ± 14.66 | 331.14 ± 65.98 |
| | mice | | | | | |
| 10 | Model | L3-8 | 10 | 68.54 ± 13.23 | 48.97 ± 11.08 | 328.49 ± 53.68 |
| | mice | | | | | |
| 11 | Model | DHM | 10 | 85.25 ± 12.73 | 66.35 ± 14.97 | 380.97 ± 63.82 |
| | mice | | | | | |

According to the results in Table 6, the groups 2 to 11 have significantly higher levels of TNF-α, IL-6, and IL-8 than the group 1, and the groups 3 to 10 have significantly lower levels of TNF-α, IL-6, and IL-8 than the group 2. TNF-α, IL-6, and IL-8 are all inflammatory markers. The immunosuppression is an abnormal state characterized by a compromised immune function and a reduced response ability to antigens. After the model mice are injected with cyclophosphamide, the levels of TNF-α, IL-6, and IL-8 significantly increase, indicating that cyclophosphamide causes the damage to the immune system in mice. After the DHM derivative of the present disclosure is added, the levels of TNF-α, IL-6, and IL-8 are reduced significantly, indicating a regulatory role for the immune function in the body.

### Example 5 Studies on Degenerative Diseases

AD, commonly known as senile dementia, predominantly affects elderly populations at an age of 60 years or more. Clinical manifestations of AD primarily include spatial cognitive impairment, executive dysfunction, and memory decline, AD is a prevalent neurodegenerative disease. In this example, AD rats were adopted to investigate the therapeutic effects of the compounds in the present disclosure for neurodegenerative diseases.

The current studies show that the excessive deposition of Aβ is a primary cause in the pathogenesis of AD. In this study, an AD model was established through the microinjection of Aβ₁₋₄₂ into bilateral hippocampal regions of a rat.

a. Preparation of a Aβ₁₋₄₂ oligomer: 1 mg of a Aβ₁₋₄₂ monomer powder and hexafluoroisopropanol (HFIP) were pre-cooled on ice. 200 μL of HFIP was added to an EP tube with 1 mg of the Aβ₁₋₄₂ monomer powder. The EP tube was sealed, vortexed for thorough mixing, and incubated at room temperature for 60 min until a resulting solution was clear to produce a 1 mmol/L Aβ₁₋₄₂-HFIP solution. The Aβ₁₋₄₂-HFIP solution was placed on ice for 5 min. 4 sterile EP tubes were taken, and 55 μL of the Aβ₁₋₄₂-HFIP solution was dispensed in each EP tube. HFIP was allowed to completely volatilize in a fume hood to produce a colorless and transparent Aβ₁₋₄₂ peptide film, which was stored in a −20° C. freezer. Before use, a dispensed EP tube was taken and placed on an ice box for operations. 11 μL of dimethylsulfoxide (DMSO) was added, and an ultrasonic treatment was conducted for 10 min (300 W, 35 Hz) in a water bath. Then 539 μL of PBS was added, vortexing was conducted for thorough mixing, and incubation was conducted in a 4° C. freezer for 1 d. After the incubation was completed, the EP tube was placed in a centrifuge, and centrifuged at 1,000 r/min and 4° C. for 10 min. A resulting supernatant was collected to obtain a 100 μmol/L Aβ₁₋₄₂ oligomer.

b. 110 healthy male SD rats (normal rats) that were 8 to 10 week old and had a body weight of (20±20) g were selected and randomly grouped according to Table 7. Aβ₁₋₄₂ rats were constructed as follows: Rats were anesthetized, and bilateral hippocampal regions were located according to the rat brain atlas. With an anterior fontanelle as a zero point, a site at 2.4 mm lateral to a midline and 3.8 mm posterior to the anterior fontanelle, and a needle insertion depth of 3.0 mm, bilateral hippocampal regions of a rat were each injected with 2 μL of Aβ₁₋₄₂ (5 μg/μL). Resulting rats were referred to as AD rats. In a DHM derivative group, a DHM derivative was administered at 120 mg/kg. In a control group, an equal amount of normal saline was intragastrically administered. The administration lasted for 4 weeks.

c. Morris water maze: After the treatment, all rats were subjected to a 5 d place navigation test. Rats were placed in a water tank from four different quadrants. A time taken for rats in each group to find and stand on a platform was recorded. If a platform was not found within 60 s, an escape latency was recorded as 60 s, and a rat needed to be guided to the platform to rest for 10 s before the subsequent experiment was conducted. After a 24 h interval, the platform was hidden, and a spatial probe test was conducted. Rats of each group were allowed to enter the water maze from a quadrant opposite to a quadrant of the original platform. The following parameters were recorded within 60 s: a movement trajectory of rats, a number of crossings over the original platform, and a proportion of a time spent in a target quadrant in the total time.

TABLE 7
Influence of DHM derivatives 1 to 8 on the memory impairment in mice

| | | | | | | | | | Proportion | |
|---|---|---|---|---|---|---|---|---|---|---|
| | | | | | | | | | of a time | Number |
| | | | | | | | | | spent in a | of |
| | | Component | | Escape latency (s) | | | | | target | crossings |
| | | intragastrically | Number | Day | Day | Day | Day | Day | quadrant | over a |
| Group | Rat | administered | of rats | 1 | 2 | 3 | 4 | 5 | (%) | platform |
| 1 | Normal | Normal saline | 10 | 52 | 25 | 20 | 18 | 15 | 38.75 | 6 |
| | rats | | | | | | | | | |
| 2 | AD | Normal saline | 10 | 52 | 38 | 28 | 25 | 22 | 18.69 | 2 |
| | rats | | | | | | | | | |
| 3 | AD | L3-1 | 10 | 52 | 28 | 22 | 18 | 15 | 34.55 | 5 |
| | rats | | | | | | | | | |
| 4 | AD | L3-2 | 10 | 52 | 28 | 22 | 18 | 15 | 33.83 | 5 |
| | rats | | | | | | | | | |
| 5 | AD | L3-3 | 10 | 52 | 29 | 23 | 19 | 15 | 35.95 | 4 |
| | rat | | | | | | | | | |
| 6 | AD | L3-4 | 10 | 52 | 27 | 23 | 18 | 16 | 32.87 | 5 |
| | rats | | | | | | | | | |
| 7 | AD | L3-5 | 10 | 52 | 28 | 22 | 18 | 15 | 33.56 | 4 |
| | rats | | | | | | | | | |
| 8 | AD | L3-6 | 10 | 52 | 28 | 22 | 18 | 15 | 34.98 | 5 |
| | rats | | | | | | | | | |
| 9 | AD | L3-7 | 10 | 52 | 29 | 22 | 18 | 15 | 33.57 | 5 |
| | rats | | | | | | | | | |
| 10 | AD | L3-8 | 10 | 52 | 28 | 22 | 18 | 15 | 32.69 | 4 |
| | rats | | | | | | | | | |
| 11 | AD | DHM | 10 | 52 | 32 | 24 | 20 | 16 | 28.73 | 3 |
| | rats | | | | | | | | | |

It can be seen that, compared to the normal rats+normal saline group, in the AD rats+normal saline group, after 4 d of place navigation training, an escape latency is significantly extended (P<0.05), and a number of crossings over the original platform and a proportion of a time spent in a target quadrant significantly decrease (P<0.01). Compared with the AD rats+normal saline group, in the AD rats+DHM derivative group, an escape latency is significantly shortened (P<0.05), and a number of crossings over the original platform and a proportion of a time spent in a target quadrant significantly increase (P<0.05). Moreover, the derivatives 1 to 8 demonstrate relatively prominent effects. It can be known that the DHM derivatives of the present disclosure can reduce the deposition of Aβ₁₋₄₂ to play a neuroprotective role against AD, and can improve the cognitive function in rats.

d. Preparation of brain tissue specimens: After the water maze test was completed, rats were randomly selected from each group, intraperitoneally injected with 3.5% chloral hydrate at 3 mL/kg for deep anaesthetization, and subjected to cardiac perfusion with normal saline. The rapid decapitation was conducted on ice to collect a brain tissue. A half of the brain tissue was placed in a −80° C. ultra-low temperature freezer for subsequent protein detection in molecular biology. The other half of the brain tissue was added to a 4% paraformaldehyde solution for fixation, placed overnight in a 4° C. freezer, and then dehydrated with 10%, 20%, and 30% gradient sucrose solutions.

e. ELISA analysis: A rat brain tissue was selected from each group. According to an operational method in a manual, a protein extraction buffer from an ELISA kit was added, and the mouse brain tissue was fully ground and allowed to stand for 3 h to achieve residue sedimentation. A resulting supernatant was collected, tested for a concentration, and diluted with an EIA buffer as needed. An antibody was labeled. Standards were prepared, and a standard curve was plotted. A sample was loaded, and incubation was conducted at 4° C. for 12 h. A plate was washed, and a 3,3′,5,5′-tetramethylbenzidine (TMB) chromogenic solution was added for color development. The absorbance was determined by a microplate reader.

(1) ELISA results of Aβ₁₋₄₂ in rat brains were shown in FIG. 1. According to the results, there is a significantly higher level of Aβ₁₋₄₂ in rat brains of the group 2 than in rat brains of the group 1, and there is a significantly-lower expression level of Aβ₁₋₄₂ in rat brains of the groups 3 to 10 than in rat brains of the group 2.

f. Semi-quantitative detection of protein levels (Western Blotting (WB)):

### (1) Protein Extraction

About 200 mg of a brain tissue collected from each group was taken out from the ultra-low temperature freezer and weighed. A protein lysis buffer was then added at 10 mL/g and phenylmethanesulfonyl fluoride (PMSF) was added at 20 μL/g, and thorough mixing was conducted. The tissue was ground by a handheld homogenizer to produce a particle-free homogenous suspension. The particle-free homogenous suspension was allowed to stand on ice for 10 min, and then centrifuged for 15 min in a 4° C. low-temperature high-speed centrifuge at 12,000 rpm, and a resulting supernatant was collected and stored.

(2) Protein concentration detection: A protein concentration in each group was detected with a Beyotime BCA kit, and a protein concentration in each group was equilibrated.

(3) Protein denaturation: A protein sample and a sodium dodecyl sulfate-polyacrylamide gel electrophoresis (SDS-PAGE) loading buffer were thoroughly mixed in a ratio of 4:1, heated in a constant-temperature metal bath at 100° C. for 5 min, cooled naturally, and then stored in a −20° C. freezer for later use.

(4) Gel preparation: A separation gel at a desired concentration was prepared according to a manual.

(5) Electrophoresis: The prepared separation gel was fixed on a gel cassette and placed in an electrophoresis tank, and an electrophoresis buffer was added. A comb was taken out, a protein sample was loaded, and the electrophoresis tank was covered with a lid. A voltage was adjusted to 80 V, and electrophoresis was started. When it was observed that a Marker protein was separated, the voltage was adjusted to 120 V. Once it was observed that a target protein was separated, the electrophoresis was paused for electrotransfer.

(6) Electrotransfer: A gel band in a desired protein range was cut according to the Marker and placed on a transfer frame, and a polyvinylidene fluoride (PVDF) membrane was closely attached to the gel band. The transfer frame was placed in an electrophoresis tank, and a current was adjusted to 250 mA. Electrotransfer time was determined based on a molecular weight of a target protein.

(7) Blocking: After the electrotransfer was completed, a resulting PVDF membrane was taken out and blocked in a 5% bovine serum albumin (BSA) blocking solution at 37° C. for 2 h.

(8) Incubation with a primary antibody: A resulting membrane was incubated in the primary antibody diluted evenly at 4° C. for 12 h.

(9) Incubation with a secondary antibody: A resulting membrane was taken out, and washed 3 times with 1×PBST for 10 min each time, and then incubated in a secondary antibody of a corresponding species at 37° C. for 1 h.

(10) Development: A resulting membrane was taken out and washed 3 times with 1×PBST for 20 min each time. An enhanced chemiluminescence (ECL) ultra-sensitive developing solution (solution A:solution B=1:1) was added dropwise. A resulting membrane was tested by the Image Lab software for a grayscale value.

### Influence of DHM on Autophagy-Related Proteins in Rats

Beclin1 plays a key role in the autophagy, and is an essential molecule for autophagosome generation. Beclin1 can recruit a plurality of autophagy-related proteins to regulate the production and maturation of autophagosomes. Beclin1 can reflect the induction level for autophagy. LC3 and p62 are classical markers for autophagy, which are crucial for autophagy initiation, nucleation, and expansion. LC3-II is a structural protein for autophagosomes. A level of LC3-II reflects a quantity of activated autophagosomes. A LC3-II/I ratio is commonly used to measure an autophagy level. p62 is a marker protein reflecting an autophagy activity. When the autophagy occurs, the p62 protein is continuously degraded in the cytoplasm. When the autophagy activity is weakened or the autophagy function is defective, the p62 protein will constantly accumulate in the cytoplasm. A content of the p62 protein indirectly reflects the autolysosome clearance level. LAMP1 is a lysosomal membrane protein. An expression level of LAMP1 reflects a quantity of lysosomes. Cathepsin D is one of the major proteolytic enzymes for lysosomes, and can degrade autolysosomes to maintain cellular homeostasis.

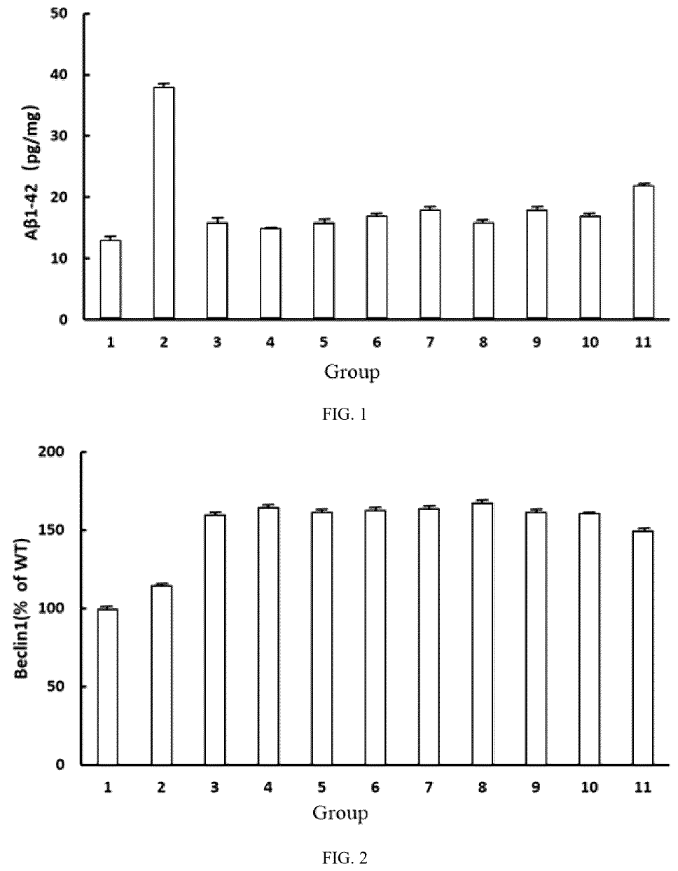

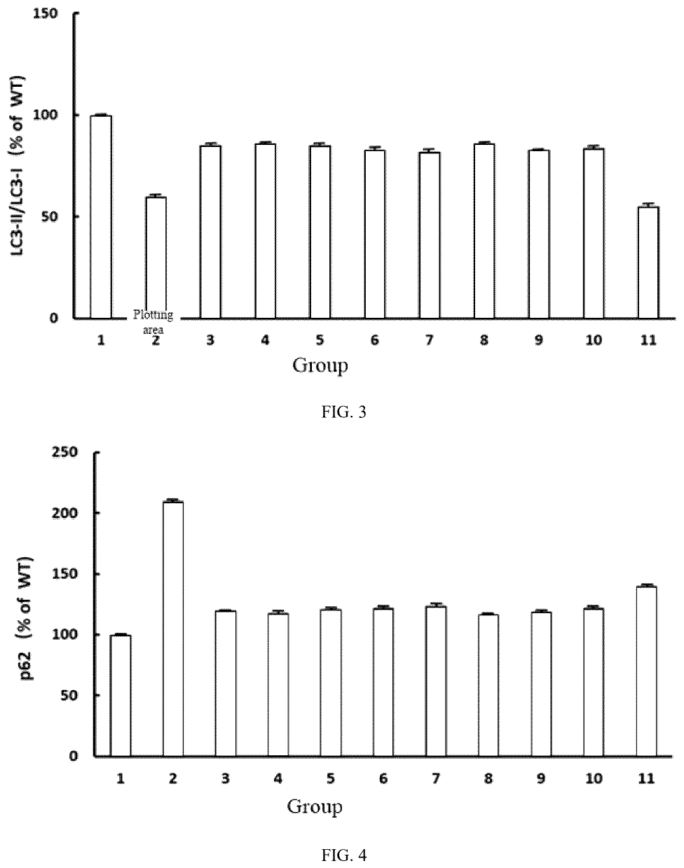

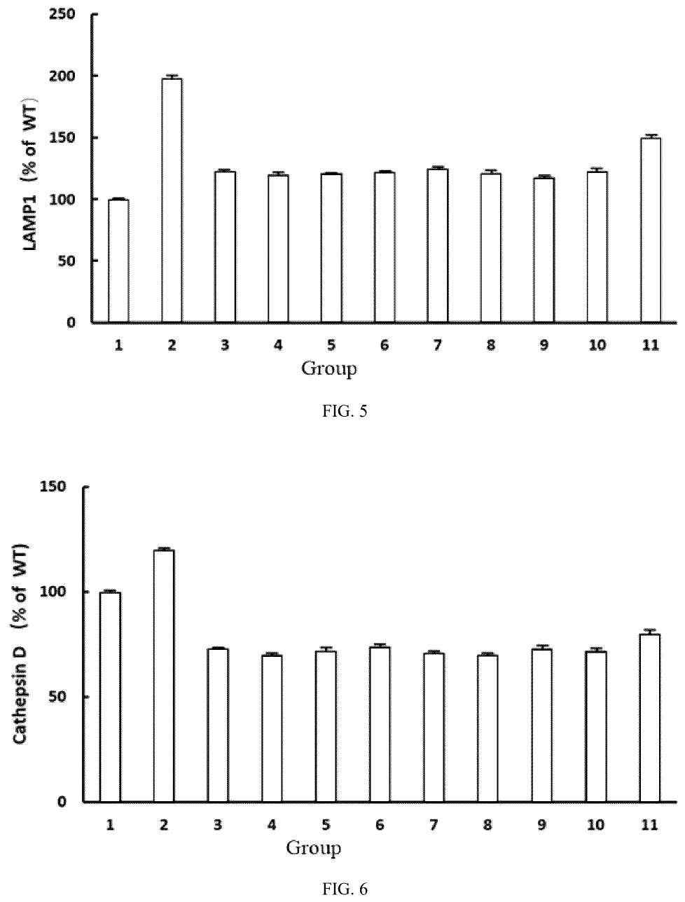

Results were shown in FIG. 2 to FIG. 6. Compared with the group 1, in the group 2, an expression level of the autophagy regulatory gene Beclin1 does not significantly change (P>0.05), an autophagy-specific marker molecule LC3-II/I ratio significantly decreases (P<0.001), expression levels of the autophagy substrate-related protein p62 and the lysosome-related membrane protein LAMP1 significantly increase (P<0.001), and an expression level of the cathepsin D increases (P<0.05). Compared with the group 2, in the groups 3 to 10, an expression level of Beclin1 increases (P<0.01), an expression level of LC3-II/I (P>0.05) does not change significantly, expression levels of p62 and LAMP1 decrease significantly (P<0.001), and an expression level of Cathepsin D decreases (P<0.01).

According to the above results, the autophagic flux initiation and autolysosome degradation functions in AD rats are impaired, and the intervention with DHM can effectively improve the autophagic flux disorder in AD rats and significantly enhance the degradation function of autolysosomes in AD rats. In summary, the DHM derivatives of the present disclosure can promote the degradation of autophagic substrates, prevent the accumulation of autolysosomes to restore the smoothness of autophagic flux, and reduce the deposition of Aβ in brains of AD mice, thereby ultimately achieving a therapeutic effect for AD.

### Example 6 Anti-Aging Test

100 male C57BL/6 mice at an age of 22 months to 24 months were adaptively raised in an animal room for 2 weeks, during which the light was adjusted according to a 12 h light/dark rhythm. During the raising, mice had free access to water and food. In a raising environment, a temperature was always maintained at 20° C. to 22° C., and a relative humidity was maintained at 50% to 60%.

Two weeks later, the mice were divided into a control group and experimental groups 1 to 9, with 10 mice in each group. In the experimental groups, the DHM and the DHM derivatives 1 to 8 were injected at 2 mg/kg/d, respectively. In the control group, an equal amount of normal saline was intraperitoneally injected. In each group, the administration was conducted once a day consecutively for 8 weeks. A body weight and a water intake were monitored three times a week.

8 weeks later, mice were sacrificed, and liver tissue samples were collected and weighed. A specified amount of PBS at a pH of 7.4 was added to a sample, and the sample was quickly frozen with liquid nitrogen and stored for later use. After a frozen sample was thawed, a temperature of the sample should still be maintained at 2° C. to 8° C. A specified amount of PBS (pH 7.4) was added, and a sample was thoroughly homogenized manually or by a homogenizer. Centrifugation was conducted for about 20 min (2,000 rpm to 3,000 rpm), and a resulting supernatant was carefully collected and dispensed. One aliquot was used for testing, and the remaining aliquots were frozen for later use. The mouse silencing regulatory factor 2-like protein 3 (SIRT3) was tested according to operational steps of an SIRT3 ELISA kit (Abcam, USA).

$$Improvement rate for an expression level of an SIRT 3 protein = ( expression level of an experimental group - an expression level of a control group ) / expression level of the control group * 100 \% .$$

TABLE 8
Improvement rates for the expression level of the SIRT3 protein

| Group | Improvement rate |
|---|---|
| Experimental group 1 (L3-1) | 20.4% |
| Experimental group 2 (L3-2) | 19.3% |
| Experimental group 3 (L3-3) | 18.5% |
| Experimental group 4 (L3-4) | 18.8% |
| Experimental group 5 (L3-5) | 19.7% |
| Experimental group 6 (L3-6) | 17.5% |
| Experimental group 7 (L3-7) | 18.1% |
| Experimental group 8 (L3-8) | 17.8% |
| Experimental group 9 (DHM) | 12.6% |

Sirtuins, as a longevity factor, are NAD⁺-dependent deacetylases, which play a pivotal role in cellular metabolism and environmental stress. The up-regulation of the expression of the SIRT3 gene can protect mitochondria from oxidative stress while enhancing the cellular energy, thereby delaying the cellular senescence. The DHM derivatives prepared in the examples of the present disclosure can improve the expression level of the SIRT3 protein in a tissue, and can demonstrate significantly superior efficacy to DHM.

Although specific examples have be described with reference to the disclosed embodiments and illustrations, such examples are merely illustrative, and do not limit the scope of the present disclosure. Modifications and variations may be made by those of ordinary skill in the art without departing from the extensive aspects of the present disclosure that are defined by the appended claims.

All publications, patents, and patent documents are incorporated herein by reference as if individually incorporated herein by reference. It should not be construed accordingly as a limitation inconsistent with the present disclosure. The present disclosure has been described with reference to various specific and preferred embodiments and techniques. However, it should be understood that numerous variations and modifications may be made within the spirit and scope of the present disclosure.

What is claimed is:

1. A dihydromyricetin (DHM) derivative or a pharmaceutically acceptable salt thereof, wherein the DHM derivative is a compound shown in a formula I: 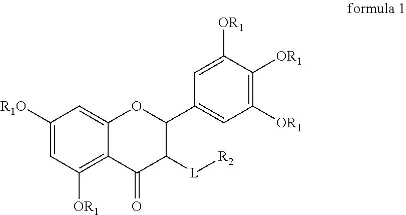
wherein R₁ is independently selected from C₁₋₆ alkyl, C₁₋₆ alkylcarbonyl, C₁₋₆ alkylsulfonyl, C₁₋₆ alkylsulfinyl, or H;
L is selected from —(CR₃R₄)n-C(═X)—, —C(═X)—(CR₃R₄)n-, —(CR₃R₄)m-, —(CR₃R₄)n-C(═O)—NH—, —(CR₃R₄)n-NH—C(═O)—, —C(═O)—NH—(CR₃R₄)n-, —NH—C(═O)—(CR₃R₄)n-, —(CR₃R₄)n-C(═O)—NH—(CR₃R₄)n-, —(CR₃R₄)n-NH—C(═O)—(CR₃R₄)n-, or O—C(═O)—(CR₃R₄)n-, wherein X is selected from O, S, or NH; n and m are each independently selected from integers of 0 to 6; and
R₃ and R₄ are each independently selected from H, C₁₋₆ alkyl, a halogen, hydroxyl, —NR₅R₆, or —CN;
R₂ is selected from 3- to 12-membered heterocyclyl, wherein the 3- to 12-membered heterocyclyl is optionally substituted with C₁₋₆ alkyl, a halogen, hydroxyl, —NR₅R₆, —CN, —SF₅, or C₁₋₆ alkoxy; or
R₂ is selected from —NR₅R₆,
wherein R₅ and R₆ are each independently selected from H, C₁₋₆ alkyl, or —C(═NH)NH₂.

2. The DHM derivative or the pharmaceutically acceptable salt thereof according to claim 1, wherein R₁ is independently selected from C₁₋₆ alkyl or H; and L is selected from —(CR₃R₄)m-, wherein R³ and R₄ are each independently selected from H and C₁₋₆ alkyl.

3. The DHM derivative or the pharmaceutically acceptable salt thereof according to claim 1, wherein R₂ is selected from 5- to 10-membered heterocyclyl, wherein the 5- to 10-membered heterocyclyl is optionally substituted with C₁₋₆ alkyl, a halogen, hydroxyl, —NR₅R₆, —CN, or C₁₋₆ alkoxy; or
R₂ is selected from —NR₅R₆, wherein R⁵ and R₆ are each independently selected from H, C₁₋₆ alkyl, and —C(═NH)NH₂.

4. The DHM derivative or the pharmaceutically acceptable salt thereof according to claim 3, wherein the C₁₋₆ alkyl is selected from methyl, ethyl, n-propyl, isopropyl, n-butyl, isobutyl, tert-butyl, n-pentyl, isopentyl, neopentyl, or n-hexyl.

5. The DHM derivative or the pharmaceutically acceptable salt thereof according to claim 3, wherein the 5- to 10-membered heterocyclyl is selected from azacyclobutyl, oxacyclobutyl, tetrahydrofuranyl, dioxolyl, pyrrolidinyl, imidazolidinyl, pyrazolidinyl, pyrrolinyl, tetrahydropyranyl, piperidinyl, morpholinyl, dithianyl, thiomorpholinyl, piperazinyl, trithianyl, or diazepanyl.

6. The DHM derivative or the pharmaceutically acceptable salt thereof according to claim 1, wherein the compound shown in the formula I is selected from the following structures:

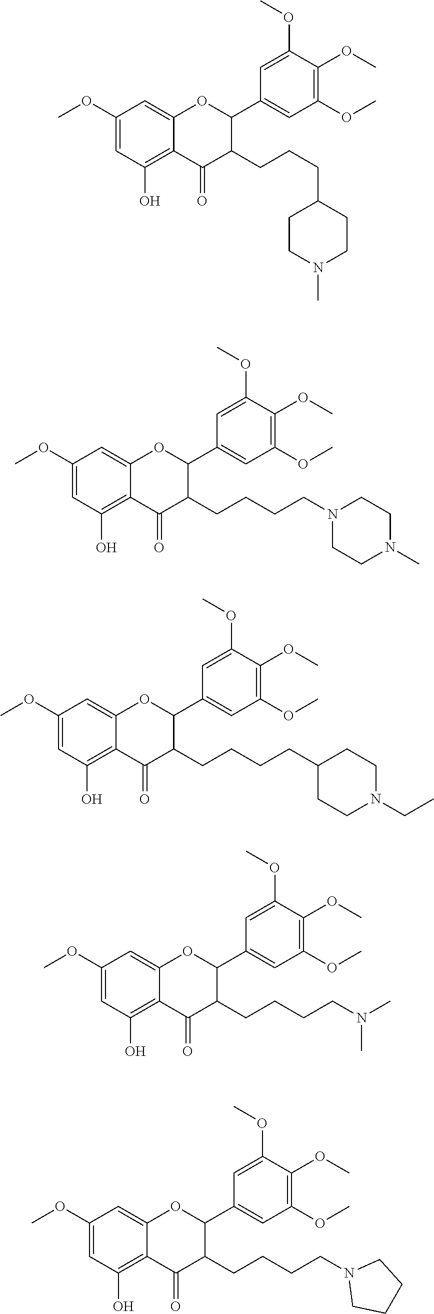

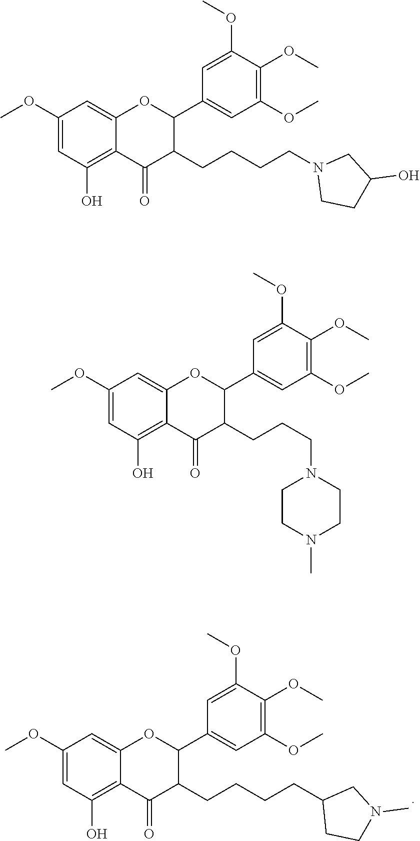

7. A preparation method of the DHM derivative according to claim 1, comprising the following step: 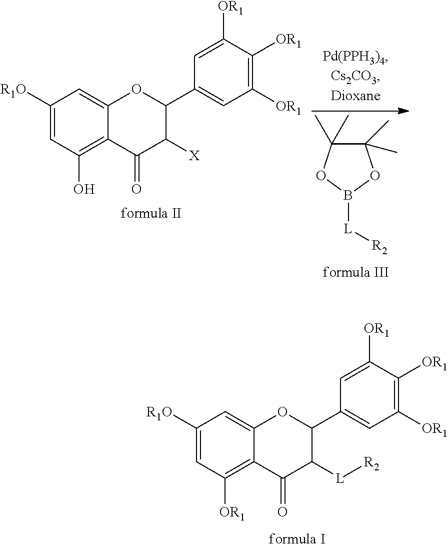
allowing a compound shown in a formula II to react with a compound shown in a formula III to produce the compound shown in the formula I, wherein X₁ is selected from Br, I, or OTf.

8. A pharmaceutical composition comprising the DHM derivative or the pharmaceutically acceptable salt thereof according to claim 1.

9. A pharmaceutical preparation comprising the DHM derivative or the pharmaceutically acceptable salt thereof according to claim 1, and a pharmaceutically acceptable carrier.

10. The pharmaceutical preparation according to claim 9, wherein the pharmaceutically acceptable carrier comprises a pharmaceutically acceptable adjuvant.

11. The pharmaceutical preparation according to claim 10, wherein the pharmaceutically acceptable adjuvant is selected from one or more of a filler, a disintegrant, a binder, a lubricant, a surfactant, a corrigent, a wetting agent, a pH regulator, a solubilizer or cosolvent, or an osmotic pressure regulator.

12. The pharmaceutical composition according to claim 8, further comprising one or more therapeutic agents.

13. A method for treating insomnia, a sleep disorder, anxiety, cognitive decline, memory impairment, and a neurodegenerative disease, comprising administering the DHM derivative or the pharmaceutically acceptable salt thereof according to claim 1 to a subject.

14. The method according to claim 13, wherein the neurodegenerative disease comprises neuroinflammation, Alzheimer's disease (AD), Parkinson's disease, Huntington's disease, or amyotrophic lateral sclerosis.

15. A method for treating an immune system disorder, comprising administering the DHM derivative or the pharmaceutically acceptable salt thereof according to claim 1 to a subject.

16. The method according to claim 15, wherein the immune system disorder comprises rheumatoid arthritis, muscular dystrophy, systemic lupus erythematosus, spinal cord syndrome, multiple sclerosis, systemic sclerosis, scleroderma, paraneoplastic syndromes of small cell lung cancer (SCLC), renal tuberculosis, lymphoma, hepatitis B, or chronic lymphocytic leukemia.

17. A method for anti-aging, comprising administering the DHM derivative or the pharmaceutically acceptable salt thereof according to claim 1 to a subject.

18. The DHM derivative or the pharmaceutically acceptable salt thereof according to claim 2, wherein R₂ is selected from 5- to 10-membered heterocyclyl, wherein the 5- to 10-membered heterocyclyl is optionally substituted with C₁₋₆ alkyl, a halogen, hydroxyl, —NR₅R₆, —CN, or C₁₋₆ alkoxy; or
R₂ is selected from —NR₅R₆, wherein R₅ and R₆ are each independently selected from H, C₁₋₆ alkyl, and —C(═NH)NH₂.

19. The DHM derivative or the pharmaceutically acceptable salt thereof according to claim 2, wherein the compound shown in the formula I is selected from the following structures:

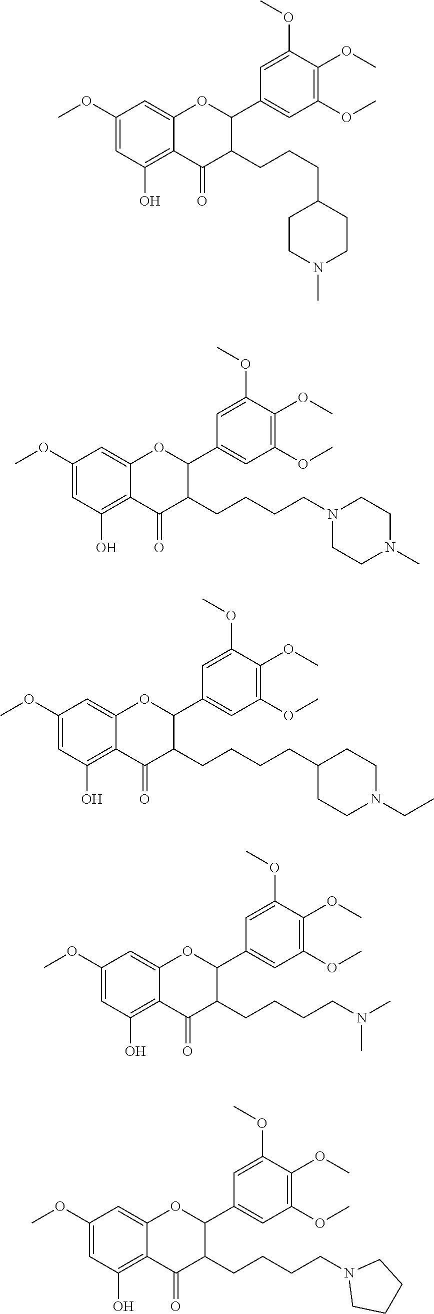

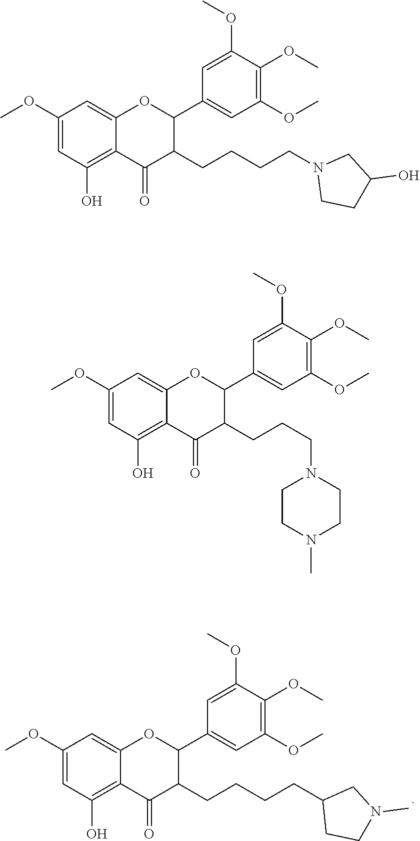

20. The DHM derivative or the pharmaceutically acceptable salt thereof according to claim 3, wherein the compound shown in the formula I is selected from the following structures: 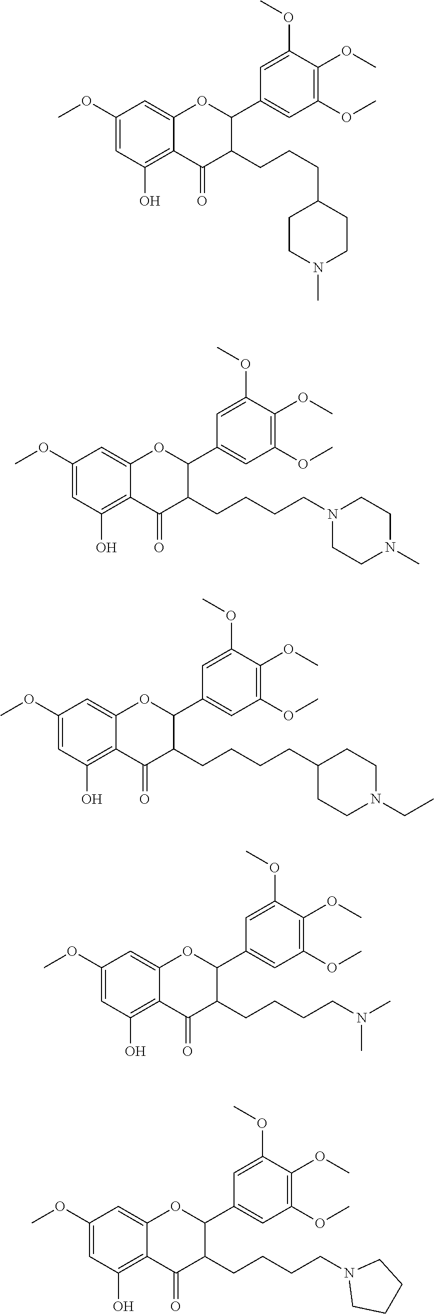 [chemical structure]
